(19) United States
(12) Patent Application Publication
(10) Pub. No.: US 2022/0113309 A1
(43) Pub. Date: Apr. 14, 2022

(54) DETECTION COMPONENT FOR BLOOD GROUP ANTIGENS

(71) Applicant: INTEC PRODUCTS, INC., Xiamen (CN)

(72) Inventor: Jinggao Hu, Xiamen (CN)

(21) Appl. No.: 17/560,741

(22) Filed: Dec. 23, 2021

Related U.S. Application Data
(63) Continuation of application No. PCT/CN2020/100784, filed on Jul. 8, 2020.

(30) Foreign Application Priority Data
Jul. 23, 2019 (CN) 201921168987.2

(51) Int. Cl. G01N 33/558; G01N 33/68; G01N 33/543

(52) U.S. Cl. CPC G01N 33/558; G01N 2800/24; G01N 33/54387; G01N 33/6854

(57) ABSTRACT

A detection component for blood group antigens comprising a test strip and a sample addition device, and a sample is transported to a testing section of the test strip by a sampling ring of the sample addition device. Immunochromatography technology is used to identify human blood group antigens with existing blood group antibodies. The antibodies are pre-coated with and solidified on the reaction membrane to define testing points and then assembled to the flushing pad, and the water absorption paper and the pad to define the test strip.

### RELATED APPLICATIONS

The present disclosure is a continuation of International Patent Application PCT/CN2020/100784, filed on Jul. 8, 2020, which claims priority to Chinese Patent Application 201921168987.2, filed on Jul. 23, 2019. International Patent Application PCT/CN2020/100784 and Chinese Patent Application 201921168987.2 are incorporated herein by reference.

### FIELD OF THE DISCLOSURE

The present disclosure relates to a detection component for blood group antigens.

### BACKGROUND OF THE DISCLOSURE

Blood groups of red blood cells are classified according to surface antigens of the red blood cells in the blood. According to various types of blood group antigens, the International Society of Blood Transfusion (ISBT) reported that there are 29 groups and 221 antigens in 2004, and each blood group is independently inherited. The ABO blood group and the RhD blood group are two of the most important blood groups in humans and have important clinical implications due to high immunogenicity, especially closely related to clinical blood transfusion, organ transplantation, etc. Correct blood group testing is important work to ensure safety of the clinical blood transfusion and rescue of life. At present, tests of the ABO blood group and the RhD blood group are routine blood group serological test items necessary before blood transfusion.

In the existing techniques, the commonly used clinical testing methods for blood group antigens comprise an agglutination method, a microcolumn gel method, and a solid-phase card method. Reagents used in the agglutination method need refrigerated transport at 2-8° C., the results cannot be standardized without control reagents, and a centrifuge or a glass slide is further required for the reaction. The operation is cumbersome, the results are greatly affected by subjective deviations, easily leading to errors in the blood group testing, and there might be problems, by way of example, contamination possibilities of liquid reagents and blood samples. In the microcolumn gel method, special gel cards are required. The costs of gel and blank card are relatively high. Further, a sample to be tested needs to be pre-processed to be configured into an about 1% red blood cell suspension. At the same time, a special centrifuge is required, and the reaction time is long, usually 5-10 minutes. Therefore, it has disadvantages of complicated operation, time-consuming, labor-intensive, cost-consuming, etc. Blood group antigen testing using the solid-phase card method is a newly emerged product in recent years. The main problem of the solid-phase card method is that there is no control; the results cannot be standardized and are greatly affected by subjective deviations, the card is large in size, a lot of medical wastes are easily created, and the cost is high. When the blood group antigens are tested, each test strip is relatively independent. When various blood group antigens are tested, it is necessary to separately add samples to a sample addition section of each test strip and to separately add flushing liquid to a flushing section of each test strip. Users need to drip samples or flush liquids in multiple sections. The operation is complicated and is prone to errors. At the same time, the sample volume is large.

In sum, the testing methods for the blood group antigens in the existing techniques have problems, by way of example, high manual labor intensity, long testing time, high cost, the results are more affected by subjective deviations, the possibility of contamination, etc. Therefore, it is necessary to make substantial improvements to traditional testing methods for blood group antigens.

### BRIEF SUMMARY OF THE DISCLOSURE

An objective of the present disclosure is to provide a detection component for blood group antigens to overcome deficiencies of existing techniques.

A technical solution of the present disclosure is as follows.

A detection component for blood group antigens comprises:

A test strip comprising a base board, wherein a reaction membrane, a flushing pad, a water absorption pad, and a sealing pad are disposed on the base board, the reaction membrane is disposed on a middle portion of the base board, one end of the flushing pad overlaps one end of the reaction membrane, one end of the water absorption pad overlaps another end of the reaction membrane, one part of the sealing pad is stuck to the reaction membrane, another part of sealing pad is stuck to the flushing pad, a section of the reaction membrane disposed between one end of the sealing pad and the one end of the water absorption pad defines a testing section, an A testing point, a B testing point, a D testing point, and a Ctl testing point are disposed on the testing section, the A testing point is coated with a solidified anti-A monoclonal antibody, the B testing point is coated with a solidified anti-B monoclonal antibody, the D testing point is coated with a solidified anti-D monoclonal antibody, the Ctl testing point is coated with a solidified anti-RBC monoclonal antibody;

A sample addition device comprising a handle and a sampling ring, wherein the sampling ring is disposed on one end of the handle, a length of the sampling ring is less than a width of the reaction membrane and is sufficient to cover the A testing point, the B testing point, the D testing point and the Ctl testing point;

An inner side wall of the sampling ring comprises a plurality of protrusions equally spaced along a circumference of the inner side wall of the sampling ring to increase a surface tension force of the sampling ring and to ensure a sampling volume of 10-20 μL; and

A sample is transported to the testing section of the test strip by the sampling ring of the sample addition device.

In a preferred embodiment of the present disclosure, a tag is disposed on the water absorption pad.

In a preferred embodiment of the present disclosure, the A testing point, the B testing point, the D testing point and the Ctl testing point are linearly arrayed or staggered up and down to be shaped as an “N” along a width direction of the testing section.

In a preferred embodiment of the present disclosure, the sampling ring is shaped as an ellipse or a rectangle.

In a preferred embodiment of the present disclosure, the sampling ring is shaped as a rectangle having a circular angle.

In a preferred embodiment of the present disclosure, the reaction membrane is a nitrocellulose membrane, a cellulose acetate membrane, a polyethylene membrane, or a nylon membrane.

In a preferred embodiment of the present disclosure, the reaction membrane is a nitrocellulose membrane having a back support, having a capillary flow rate of less than 90 seconds, and a width of 4 cm.

The present disclosure has the following advantages.

1. The present disclosure uses immunochromatography technology to identify human blood group antigens with existing blood group antibodies. The antibodies are pre-coated and solidified on the reaction membrane to define testing points and then assembled to the flushing pad, the water absorption pad, and the base board to define the test strip. When a sample is tested (e.g., anticoagulated whole blood, blood from a fingertip, a suspension of red blood cells), the sample is dripped on the reaction membrane, and the flushing solution for the sample is then dripped on the flushing pad. When the red blood cells in the sample to be tested are reacted with the antibodies of a solid-phase due to immunoconjugate, the red blood cells are retained at the testing points disposed on the reaction membrane and define a red dot, which indicates a positive reaction and indicates that surfaces of the red blood cells have the correlated blood group antigens. When antigen-antibody immune response does not occur, the red blood cells cannot be retained and move forward into the water absorption pad due to chromatography function, the testing points disposed on the reaction membrane are white in color, and only the positive control point (the Ctl testing point) is red in color, which indicates a negative reaction.

2. The design is compact, the integration is high, the volume is small, and the product cost is low, a test for the ABO blood group is completed using one drop of sample, manual operation is simplified during testing, false testing caused by manual errors is reduced, and medical waste is less due to no card sleeve design.

3. In the present disclosure, the transportation and the storage are convenient. The detection component for blood group antigens is stored at room temperature (2-30° C.), the validity period is long (24 months), the sensitivity is high, the detection component for blood group antigens is a solid-phase test strip and is portable, the testing time is shortened to 1 minute, the operation is simple, positive control is provided, interpretation of results is easy, objective and reliable, and no auxiliary equipment is required.

### BRIEF DESCRIPTION OF THE DRAWINGS

FIG. 1 illustrates a structural view of a test strip of the present disclosure.

FIG. 2 illustrates a structural view of a sample addition device of the present disclosure.

### DETAILED DESCRIPTION OF THE EMBODIMENTS

The present disclosure will be further described in combination with the accompanying embodiments and drawings.

A detection component for blood group antigens comprises a test strip 1 and a sample addition device 2.

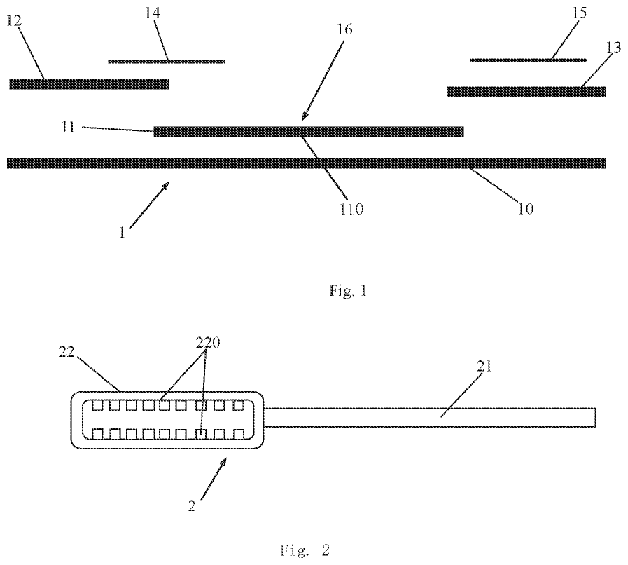

Referring to FIG. 1, the test strip 1 comprises a base board 10, and a reaction membrane 11, a flushing pad 12, a water absorption pad 13, and a sealing pad 14 are disposed on the base board 10. The reaction membrane 11 is disposed on a middle portion of the base board 10 (i.e., a loader), and one end of the flushing pad 12 overlaps one end of the reaction membrane 11. One end of the water absorption pad 13 overlaps another end of the reaction membrane 11, and a tag 15 is stuck to an upper side of the water absorption pad 13. One part of the sealing pad 14 is stuck to the reaction membrane 11, and another part of the sealing pad 14 is stuck to the flushing pad 12. A section of the reaction membrane 11 disposed between one end of the sealing pad 14 and the one end of the water absorption pad 13 defines a testing section 16. The testing section 16 integrates an observation window and a sample addition section on which an A testing point, a B testing point, a D testing point, and a Ctl testing point are disposed. The A testing point is coated with a solidified anti-A monoclonal antibody and defines a light cyan color. The B testing point is coated with a solidified anti-B monoclonal antibody and defines a light yellow color. The D testing point is coated with a solidified anti-D monoclonal antibody and defines a light red color. The Ctl testing point is coated with a solidified anti-RBC monoclonal antibody and defines a light blue color. The A testing point, the B testing point, the D testing point, and the Ctl testing point are linearly arrayed or staggered up and down to be shaped as an “N” along a width direction of the testing section 16.

The reaction membrane 11 is a nitrocellulose membrane, a cellulose acetate membrane, a polyethylene membrane, or a nylon membrane. Preferably, the reaction membrane 11 is a nitrocellulose membrane having a back support 110, having a capillary flow rate of less than 90 seconds, and a width of 4 cm, and a pore size of the nitrocellulose membrane is preferably selected such that a single red blood cell can penetrate into the porous structure and move chromatographically.

The flushing pad 12 is made of porous polymer material having water conductivity and hydrophilicity, preferably, glass fiber.

The water absorption pad 13 is made of absorbent materials, preferably, cotton pulp paper.

Referring to FIG. 2, the sample addition device 2 comprises a handle 21 and a sampling ring 22. The sampling ring 22 is disposed on one end of the handle 21. A length of the sampling ring 22 is less than a width of the reaction membrane 11 and is sufficient to cover the A testing point, the B testing point, the D testing point, and the Ctl testing point. An inner side wall of the sampling ring 22 comprises a plurality of protrusions 220 equally spaced along a circumference of the inner side wall of the sampling ring 22 to increase a surface tension force of the sampling ring 22 and to ensure a sampling volume of 10-20 μL. The handle 21 is solid, and a cross-section of the handle 21 can be circular or polygonal, preferably, circular.

In a preferred embodiment, a shape of the sampling ring 22 defines a rectangle shape having circular angles.

A sample is transported to the testing section 16 of the test strip 1 by the sampling ring 22 of the sample addition device 2.

A method of the present disclosure is applied as follows: the sample to be tested (e.g., anticoagulated whole blood, blood from a fingertip, a suspension of red blood cells) is added to the testing section 16 by the sampling ring 22, a flushing solution is added to the flushing pad 12, and results are observed after about 1 minute. When the red blood cells in the sample to be tested are reacted with the antibodies pf the solid-phase by immunoconjugate, the red blood cells are retained on the testing points on the reaction membrane 11 (i.e., points pre-coated with antibodies) and define a red dot, which indicates a positive reaction and indicates that surfaces of the red blood cells comprise the correlated blood group antigens. When antigen-antibody immune response does not occur, the red blood cells cannot be retained and move forward into the water absorption pad 13 due to chromatography function, the testing points on the reaction membrane 11 are white in color, and only the positive control point (i.e., the Ctl testing point) is red in color, which indicates a negative reaction.

The aforementioned embodiments are merely some embodiments of the present disclosure, and the scope of the disclosure is not limited thereto. Thus, it is intended that the present disclosure cover any modifications and variations of the presently presented embodiments provided they are made without departing from the appended claims and the specification of the present disclosure. By way of example: 1) the solidified anti-A monoclonal antibody, the solidified anti-B monoclonal antibody, and solidified anti-D monoclonal antibody are replaced with antibodies of other blood groups; and/or 2) testing points for antibodies of other blood groups are added.

### INDUSTRIAL APPLICABILITY

The present disclosure provides a detection component for blood group antigens, which comprises a test strip and a sample addition device. A sample is transported to the testing section of the test strip by the sampling ring of the sample addition device. The present disclosure uses immunochromatography technology to identify human blood group antigens with existing blood group antibodies. The antibodies are pre-coated and solidified on the reaction membrane to define testing points and then assembled to the flushing pad, the water absorption pad, and the base board to define the test strip. When a sample is tested, the sample is dripped on the reaction membrane, and the flushing solution for the sample is then dripped on the flushing pad. When the red blood cells in the sample to be tested are reacted with the antibodies of the solid phase due to immunoconjugate, the red blood cells are retained at the testing points disposed on the reaction membrane and define a red dot, which indicates a positive reaction. When antigen-antibody immune response does not occur, the red blood cells cannot be retained and move forward into the water absorption pad due to chromatography function, the testing points disposed on the reaction membrane are white in color, and only the positive control point (the Ctl testing point) is red in color, which indicates a negative reaction. The industrial applicability is good.

What is claimed is:

1. A detection component for blood group antigens, comprising:
a test strip comprising a base board, wherein:
a reaction membrane, a flushing pad, a water absorption pad, and a sealing pad are disposed on the base board,
the reaction membrane is disposed on a middle portion of the base board,
one end of the flushing pad overlaps one end of the reaction membrane,
one end of the water absorption pad overlaps another end of the reaction membrane,
one part of the sealing pad is stuck to the reaction membrane,
another part of sealing pad is stuck to the flushing pad,
a section of the reaction membrane disposed between one end of the sealing pad and the one end of the water absorption pad defines a testing section,
an A testing point, a B testing point, a D testing point, and a Ctl testing point are disposed on the testing section,
the A testing point is coated with a solidified anti-A monoclonal antibody,
the B testing point is coated with a solidified anti-B monoclonal antibody,
the D testing point is coated with a solidified anti-D monoclonal antibody, and
the Ctl testing point is coated with a solidified anti-RBC monoclonal antibody; and
a sample addition device comprising a handle and a sampling ring, wherein:
the sampling ring is disposed on one end of the handle,
a length of the sampling ring is less than a width of the reaction membrane and is sufficient to cover the A testing point, the B testing point, the D testing point, and the Ctl testing point,
an inner side wall of the sampling ring comprises a plurality of protrusions equally spaced along a circumference of the inner side wall of the sampling ring to increase a surface tension force of the sampling ring and to ensure a sampling volume of 10-20 μL, and
a sample is transported to the testing section of the test strip by the sampling ring of the sample addition device.

2. The detection component for the blood group antigens according to claim 1, wherein a tag is disposed on the water absorption pad.

3. The detection component for the blood group antigens according to claim 1, wherein the A testing point, the B testing point, the D testing point, and the Ctl testing point are linearly arrayed or staggered up and down to be shaped as an “N” along a width direction of the testing section.

4. The detection component for the blood group antigens according to claim 1, wherein the sampling ring is shaped as an ellipse or a rectangle.

5. The detection component for the blood group antigens according to claim 1, wherein the sampling ring is shaped as a rectangle having a circular angle.

6. The detection component for the blood group antigens according to claim 1, wherein the reaction membrane is a nitrocellulose membrane, a cellulose acetate membrane, a polyethylene membrane, or a nylon membrane.

7. The detection component for the blood group antigens according to claim 6, wherein the reaction membrane is a nitrocellulose membrane having a back support, having a capillary flow rate of less than 90 seconds, and a width of 4 cm.

8. A detection kit for blood group antigens, comprising:
a test strip comprising a base board, wherein:
a reaction membrane, a flushing pad, a water absorption pad, and a sealing pad are disposed on the base board,
the reaction membrane is disposed on a middle portion of the base board,
the flushing pad is disposed on a side of the reaction membrane,
one end of the flushing pad overlaps the reaction membrane,
the water absorption pad is disposed on another side of the reaction membrane,
one end of the water absorption pad overlaps another end of the reaction membrane,
one part of the sealing pad is stuck to the reaction membrane,
another part of sealing pad is stuck to the flushing pad,
a section of the reaction membrane disposed between one end of the sealing pad and the one end of the water absorption pad defines a testing section,
an A testing point, a B testing point, a D testing point, and a Ctl testing point are disposed on the testing section,
the A testing point is coated with a solidified anti-A monoclonal antibody,
the B testing point is coated with a solidified anti-B monoclonal antibody,
the D testing point is coated with a solidified anti-D monoclonal antibody,
the Ctl testing point is coated with a solidified anti-RBC monoclonal antibody; and
a sample addition device comprising a sampling ring, wherein the sampling ring is configure to add a sample to the A testing point, the B testing point, the D testing point, and the Ctl testing point at a same time.

9. The detection kit for the blood group antigens according to claim 8, wherein the sampling ring is disposed with a handle and a plurality of protrusions equally spaced and configured to respectively correspond to the A testing point, the B testing point, the D testing point, and the Ctl testing point on the reaction membrane.

10. The detection kit for the blood group antigens according to claim 8, wherein a tag is disposed on the water absorption pad.

11. The detection kit for the blood group antigens according to claim 8, wherein the A testing point, the B testing point, the D testing point, and the Ctl testing point are linearly arrayed or staggered up and down to be shaped as an “N” along a width direction of the testing section.

12. The detection kit for the blood group antigens according to claim 9, wherein a tag is disposed on the water absorption pad.

13. The detection kit for the blood group antigens according to claim 9, wherein the A testing point, the B testing point, the D testing point, and the Ctl testing point are linearly arrayed or staggered up and down to be shaped as an “N” along a width direction of the testing section.
